(19) United States
(12) Patent Application Publication
(10) Pub. No.: US 2023/0264196 A1
(43) Pub. Date: Aug. 24, 2023

(54) DIGITAL ANALYSIS SYSTEM

(71) Applicant: digid GmbH, Mainz (DE)

(72) Inventors: Constantin VON GERSDORFF, Mainz (DE); Konstantin KLOPPSTECH, Mainz (DE); Nils KOENNE, Mainz (DE); Martin GRABELLUS, Mainz (DE)

(21) Appl. No.: 18/133,322

(22) Filed: Apr. 11, 2023

Related U.S. Application Data
(63) Continuation of application No. PCT/EP2022/057690, filed on Mar. 23, 2022.

(30) Foreign Application Priority Data
Mar. 23, 2021 (DE) 102021107256.5

(51) Int. Cl. B01L 3/00; G16H 10/40

(52) U.S. Cl. CPC B01L 3/502715; G16H 10/40; B01L 2300/023; B01L 2300/0663

(57) ABSTRACT

An analysis system is provided for analyzing a sample and includes a central evaluation unit and at least one decentralized measuring unit with a communication connection via a terminal to the evaluation unit. The measuring unit accommodates different sensor modules that are exchangeable with one another and the evaluation unit and/or the measuring unit determine the incidence and/or the concentration and/or the amount of an analyte in the sample on the basis of a measurement value measured in the sample by a sensor module. Moreover, the analysis system includes a set of different sensor modules exchangeable with one another and that differ with regard to their suitability for determining chemical or biochemical information of an analyte in a sample.

### CROSS REFERENCE TO RELATED APPLICATIONS

This application is a continuation of PCT Application No. PCT/EP2022/057690, filed Mar. 23, 2022, which claims priority to German Patent Application No. 10 2021 107 256.5, filed Mar. 23, 2021, the entire contents of each of which are hereby incorporated by reference in their entirety.

### TECHNICAL FIELD

The present invention relates to an analysis system for analyzing a sample, for example for determining chemical or biochemical information in the sample, preferably for carrying out medical measurements on the sample, for example to detect diseases or symptoms in a living being.

### BACKGROUND

Medical diagnostic devices relate to devices for medical diagnostics. Known diagnostic devices are typically embodied to detect specific diseases or symptoms and are therefore produced and used for a specific application.

To monitor and control diseases, in particular infectious diseases, having a high number of affected persons, for example during pandemics or epidemics, it is desirable to enable comprehensive and extensive diagnostic measures, by means of which a large number of persons can be tested for diseases or disease-specific symptoms. This requires carrying out a large number of tests and using a large number of diagnostic devices, which is linked to a high level of effort and costs. Compiling and evaluating the large amounts of test data thus obtained also requires extensive resources and is connected to further financial and time expenditure.

Conventional systems are described in U.S. Pat. Publication No. 2019/0239784 A1, DE Application No. 10 2016 114 134 A1, and DE Application No. 699 16 599 T2.

### SUMMARY OF THE INVENTION

It is an object of the present invention to provide an analysis system which is flexibly usable and enables a large number of chemical and biochemical analyses to be carried out efficiently and effectively.

Accordingly, an analysis system is proposed for analyzing a sample, preferably for determining chemical or biochemical information in the sample, comprising a central evaluation unit and at least one decentralized measuring unit, which has a communication connection to the evaluation unit via a terminal. The measuring unit is configured to accommodate different sensor modules which are exchangeable with one another. The evaluation unit and/or the measuring unit are embodied and configured to determine the incidence and/or the concentration and/or the amount of an analyte in the sample on the basis of a measurement value measured using a sensor module in the sample.

The determination of the incidence, the concentration, or the amount of the analyte can accordingly be carried out in the sensor module, which is to be associated in this context with the measuring unit, and/or in a further component of the measuring unit and/or in the evaluation unit. In other words, either in one of the mentioned components or in several of the mentioned components or in all of the mentioned components.

Furthermore, the analysis system is configured to carry out an analysis to determine chemical or biochemical information on the basis of measurement values measured by means of a sensor module.

Chemical or biochemical information can be, for example, disease-specific and/or injury-specific features or markers, for example the presence of bacteria or viruses. The chemical and biochemical information can also be features or markers for a general health status, for example a vitamin status.

A sample comprises in particular any type of biological sample, in particular also samples from animals.

A sample can also be a non-biological sample, for example a sample of a chemical material, in which the presence of a specific analyte is to be ascertained.

The analysis carried out by means of the analysis system relates to an analysis for determining or detecting chemical or biochemical information which the sample comprises. The determination or detection can relate, for example, to whether a specific analyte is present in the sample. The determination or detection can also relate to the concentration or amount in which the analyte is present in a sample.

The determination or detection of such information or parameters can thereupon be used for medical diagnosis. This can be carried out by the analysis system with and without a cooperation of medical personnel. The analysis to be carried out accordingly can optionally be a diagnostic analysis. In the present case, the term “diagnostic analysis” relates to the detection of diseases and symptoms, i.e., to the detection and determination of disease-specific and/or injury-specific features.

A result of the analysis, also designated in the present case as an “analysis result”, can accordingly indicate whether a specific chemical or biochemical information or a marker or indicator for the presence of a specific disease and/or specific symptoms was detected or not.

The analysis can comprise a physical examination. In other words, the analysis can take place on the body of a patient, i.e., on the body of a human, or a subject. The term “subject” relates in the present case to any type of living being, i.e., metabolic-active subjects, for example animals, plants, animal and vegetable organisms, microorganisms, in particular bacteria, algae, fungi, etc. Alternatively or additionally, the analysis can comprise an examination of a sample associated with a patient or a subject. The analysis of the sample can take place outside the body of the patient or the subject in this case. Such a sample can comprise, for example, cells and/or tissues and/or excretions of the patient or subject.

The proposed analysis system preferably forms a product platform or a construction kit, in particular with regard to its structural and/or functional design, which permits it to form different diagnostic devices and accordingly carry out different analyses. In other words, the analysis system can be configurable to enable different analyses to be carried out and thus to determine and/or detect different chemical or biochemical information or parameters, in particular different diseases or symptoms, with the aid of the analysis. Accordingly, the different types of analysis devices which can be provided by the analysis system, in particular by different configurations of the analysis system, can be configured for recognizing specific information and/or specific diseases and/or specific symptoms. According to one further embodiment, the analysis system can be configured to carry out an analysis, in particular in a configuration having a sensor module provided for this purpose, with the aim of recognizing disease-specific features, in particular a viral infection, for example an infection with a coronavirus, in particular the SARS-CoV2 virus.

This modular design, using which the analysis system is equipped with a certain degree of configurability, is at least provided in that different sensor modules which are exchangeable with one another are usable in the measuring unit. In other words, the modular design of the analysis system is enabled in that the analysis system, in particular with regard to its structural and/or functional design, is divided into individual components, also designated as component parts, building blocks, assemblies, or modules, which interact with one another via defined interfaces to fulfill a desired functionality or function. To provide a desired diagnostic function, the analysis system can be brought into a desired configuration, in which the components of the analysis system provided for fulfilling the diagnostic function are assembled along defined interfaces.

The proposed analysis system comprises as components or parts preferably at least the central evaluation unit, the at least one decentralized measuring unit, the terminal, and the at least one sensor module comprised in the measuring unit. The analysis system can comprise at least one configurable component to enable different analyses to be carried out. In other words, at least one component of the analysis system can be configurable application-specifically, in particular specifically by diagnosis. The central evaluation unit and/or the at least one decentralized measuring unit and/or the terminal and/or the used sensor module can especially be configured to be configurable with regard to an item of chemical or biochemical information to be detected, in particular to be configurable or configured specifically by disease and/or symptom, i.e., to be selected and/or adapted.

For example, a sensor module can be configurable application-specifically, in particular specifically by diagnosis, disease, and/or symptom, in such a way that a selection of sensor elements of the sensor module is performed and only this selection of sensor elements is included in the evaluation. An application-specific virtual sensor can accordingly be quasi-provided by the selection of a combination of sensor elements of the sensor module specific for the application. Each sensor element can output a separate measurement value, which is then transmitted to the decentralized measuring unit and/or the terminal and/or the central evaluation unit. In other words, if the sensor module has two sensor elements, two different measurement values are also measured and transmitted.

It is thus possible, for example, that using a sensor module in a first configuration, a different application-specific question can be answered than one of a second configuration. In this way, physically identical sensor modules can actually receive a different suitability with regard to the determination of chemical and/or biochemical information of an analyte in a sample by way of the corresponding different combination of sensor elements. Such sensor modules which comprise a plurality of different sensor elements which can be selected in different configurations in order to provide different suitabilities for determining chemical and/or biochemical information of an analyte in a sample are also understood herein as different sensor modules.

For example, the sensor module can be configurable with regard to a chemical or biochemical information to be detected, in particular configurable specifically by disease and/or symptom, by the selection of a first subgroup of sensor elements accommodated in the sensor module.

A second sensor module which is physically identical per se can then be configurable for a different chemical or biochemical information to be detected by the selection of a second subgroup of sensor elements accommodated in the sensor module differing from the first subgroup, in particular can be configurable for a different disease-specific and/or system-specific configuration.

The specific selection of the sensor elements accommodated in the sensor module is achieved, for example, in that only the sensor elements of the subgroup are contacted and no contact is established to the other sensor elements. This can be achieved, for example, via a contacting specific for the respective application - for example via a specific plug - which only contacts the sensor elements of the subgroup. The specific selection and the accompanying contacting can also be achieved via corresponding switches, which are closed only for the sensor elements of the subgroup and remain open for the other sensor elements. The switch configuration required for the respective application can be actuated via software or a fixed specification.

The selection is preferably implemented by the decentralized measuring unit, which has the corresponding switch and plug devices. The actuation of the switch and plug devices can take place from the decentralized measuring unit and/or from the central evaluation unit and/or from the terminal.

In these embodiments, in which contacting of one subgroup of the sensor elements takes place, the measurement values are only captured for the actually contacted sensor elements. Accordingly, no measurement values can be captured from the other sensor elements.

The selection of the subgroup of the sensor elements can also take place on the evaluation side, wherein measurement values are captured from all sensor elements and then only those of the subgroup for the respective specific evaluation are used by means of filtering of the measurement values.

As described above, the measuring unit is configured to accommodate different sensor modules which are exchangeable with one another. In other words, different sensor modules which are exchangeable with one another can be fastened to the measuring unit or communicatively coupled therewith. A sensor module in the meaning of the present disclosure relates to a sensor unit or detector unit which is configured to qualitatively and/or quantitatively acquire physical and/or chemical and/or biochemical and/or material properties. The properties acquired in this way are converted by the sensor module into electrical signals, which are also referred to as measurement values in the present disclosure and accordingly indicate the acquired properties.

The analysis system comprises a set of different sensor modules, which can each be coupled with the measuring unit and can be used accordingly in the analysis system. The measuring unit can be configured to accommodate precisely one or more than one sensor module simultaneously. In other words, the measuring unit can be coupled with one or multiple sensor modules simultaneously. The different sensor modules can relate to different types of sensor units. The different sensor modules are provided for detecting or determining different chemical or biochemical information with respect to an analyte in a sample, for example as a marker or indication of a disease and/or for medical symptoms, and can be used accordingly for this purpose. In other words, depending on which information, which disease, or which symptoms are to be detected by means of the analysis system, a sensor module provided for this purpose can be selected from the set of different sensor modules and connected to the measuring unit. Accordingly, the different sensor modules can differ with regard to their suitability for detecting specific chemical or biochemical information with respect to an analyte in a sample, in particular with respect to specific diseases and/or symptoms. The different sensor modules can be configured, for example, to detect different DNA, RNA, inflammation values, viruses, antigens, antibodies, bacteria, and/or other indicators or information. Alternatively or additionally, sensor modules can differ as to whether they can be used for detecting active or inactive, i.e., dead, pathogens.

At least two different analytes can also be detected essentially simultaneously in a specific sensor module, in order to be able to conclude specific clinical pictures due to the combination of specific parameters.

In this way, a modular design of the analysis system is proposed, in which the components to be used therein, in particular the sensor module, can be selected application-specifically. The proposed analysis system thus has a certain degree of flexibility with regard to its area of application, i.e., with regard to the diseases or symptoms to be detected. As a result, a product platform for carrying out diagnostic analyses is provided by the proposed analysis system which corresponds to a construction kit system, in which individual components, in particular the sensor module, can be used or exchanged application-specifically, while other components, for example the terminal or the central evaluation unit, can be used across applications. A high level of configurability may thus be achieved with low levels of expenditure and costs at the same time for both the producer and the user.

To enable that the measuring unit can be connected to different sensor modules which are exchangeable with one another, an interface for coupling the sensor modules to the measuring unit can be standardized. In other words, the interfaces on the different sensor modules can be embodied identically, in particular essentially identically. According to one embodiment, the sensor modules can be connected to the measuring unit via a structural interface. According to this embodiment, a sensor module can be fastened to the measuring unit in a connected state and can have a communication connection thereto. For this purpose, for example, a plug connection or a magnetic connection, in particular a magnetic connection aligning the sensor module relative to the measuring unit can be provided. Alternatively or additionally, the sensor modules can be coupled with one another via a wireless connection, for example, via a wireless network or a Bluetooth connection.

The at least one sensor module can be configured to carry out measurements and to capture the measurement values connected thereto, in particular in the form of raw data. Furthermore, the sensor module can be configured to further process the measurement values, which are then transmitted in the form of sensor data to the measuring unit. For this purpose, the sensor module can comprise a microchip, which is preferably arranged on a printed circuit board and can be configured to capture the measurement values and/or to generate the sensor data.

According to one embodiment, the sensor module can be configured to transmit encrypted data to the measuring unit. The sensor data can accordingly be generated in the form of encrypted data by the sensor module and passed on to the measuring unit.

The sensor data can comprise or indicate the measurement values captured by means of the sensor module, in particular in the form of raw data. Alternatively or additionally, the sensor data can comprise information on a measurement carried out by means of the sensor module. These information or data can in particular specify a measurement carried out by means of the sensor module, during which the measurement values were captured. For example, times or a date or an identification identifying and designating the measurement can thus be associated with individual measurement values in the sensor data. Alternatively or additionally, the sensor data can comprise data specifying the sensor module. For example, the sensor data can comprise an identification identifying the sensor module. The identification can be a product identification number (unique device identification), which relates to a product identification according to an identification system used worldwide for medical products. According to one embodiment, the sensor module can be configured to execute the analysis on the basis of the acquired measurement values. The sensor data can accordingly comprise or indicate an analysis result originating from the diagnostic analysis.

The analysis system comprises the at least one decentralized measuring unit. In the meaning of the present disclosure, the term “decentralized measuring unit”, also referred to as a measuring unit, is understood as a unit of the analysis system which is configured to capture and record the measurement values. The measuring unit comprises the terminal and is configured to accommodate the at least one sensor module. The proposed analysis system preferably comprises more than one decentralized measuring unit, which can be used at different locations.

According to one configuration, the measuring unit can comprise a sensor station, which can also be designated as a sensor hub or smart cable. The sensor station can have a communication connection to the terminal, for example via a connecting cable or wirelessly, in particular by means of a Bluetooth connection, a mobile radio connection, for example an LTE connection, a WLAN (Wireless Local Area Network) connection, or a LoRa (Long-Range Wide Area Network) connection. According to one configuration of the measuring unit, in which no sensor station is provided, the sensor module can have a direct communication connection to the terminal, in a corresponding manner as the sensor station to the terminal.

The sensor station can be able to be coupled with at least one sensor module. In other words, the sensor station can have a communication connection to one or to multiple sensor modules simultaneously, in particular via a structural and/or wireless interface. The sensor station can accordingly be provided for the purpose of receiving and processing the sensor data generated by a sensor module connected thereto. In a state in which the sensor station is connected to a sensor module, the sensor station can be configured to ascertain data relating to the sensor module, in particular on the basis of the received sensor data, such as the product identification number. In this way, the sensor station can identify a sensor module attached thereto and perform settings in response thereto, which are required for correct use of the sensor module and the analysis system. The settings to be performed can relate in this case to the means for carrying out the diagnostic analysis of the analysis system, comprising the sensor module.

As described above, the sensor data can be transmitted in encrypted form by means of the sensor module. The sensor station can accordingly be configured to decrypt the received sensor data. For this purpose, the sensor station can comprise a microchip, for example a security chip for data decryption.

In one further embodiment, the measuring unit, in particular the sensor station, can be configured to be operated in an initialization mode. The measuring unit, in particular the sensor station, can be configured to recognize whether a coupling to a sensor module is taking place or exists, in order to change into the initialization mode in response thereto. In the initialization mode, the measuring unit, in particular the sensor station, can identify the sensor module coupled or to be coupled thereto. This can take place on the basis of data transmitted by means of the sensor module, in particular by means of data specifying the sensor module, for example by means of the product identification number contained in the sensor data. This procedure can be used to authenticate the sensor module attached thereto. It can thus be ensured that only predetermined sensor modules are connected to the measuring unit and used for the measurements.

Furthermore, the measuring unit, in particular the sensor station, can be configured to be operated in a validation mode in which the correct status and/or operation of the sensor module attached thereto can be validated. The measuring unit, in particular the sensor station, can be configured to be operated in the validation mode after the initialization mode. The validation can take place for this purpose in dependence on the sensor module attached thereto, i.e., in dependence on the type of the sensor module. In the validation mode, the measuring unit, in particular the sensor station, can cause the sensor module to carry out a reference measurement. The reference measurement values captured by means of the sensor module during the reference measurement can thereupon be transmitted to the measuring unit, in particular the sensor station. The measuring unit, in particular the sensor station, can be configured to determine a correct operation and/or status of the sensor module in dependence on the reference measurement values. For this purpose, an evaluation of the reference values can take place. For example, the measuring unit, in particular the sensor station, can compare the ascertained reference measurement values to a predetermined value range. If the reference measurement values are within the predetermined value range, for example, the measuring unit, in particular the sensor station, can detect a correct status or operation of the sensor module. If this is not the case, the status or operation of the sensor module can be detected as incorrect and a message or warning can be output to a user of the measuring unit.

In one further embodiment, the measuring unit can be configured to query a status of the sensor module to detect whether it corresponds to a predetermined proper status. For this purpose, the sensor module can transmit data to the measuring unit which specify a status of the sensor module. These data specifying the status of the sensor module can indicate, for example, whether the sensor module has already been used for prior measurements or tests. If the data specifying the status of the sensor module indicate that the sensor module has a used status, the measuring unit can assess the status of the sensor module as improper.

Furthermore, the measuring unit can be configured not to permit a use of the sensor module and accordingly to terminate a measurement to be executed by means of the sensor module and not to carry out the analysis if the status of the sensor module attached thereto has been assessed as improper. In this way, the analysis system can ensure that the parts underlying a diagnostic analysis are authenticated and their proper status is verified before an analysis takes place.

Furthermore, the measuring unit, in particular the sensor station, can be configured to be operated in a measuring mode in which the sensor module is caused to carry out measurements and accordingly to capture the measurement values. In the measuring mode, the measuring unit, in particular the sensor station, can receive the sensor data comprising the measurement values. The measuring unit, in particular the sensor station, can be configured to be operated in the measuring mode after the validation mode. The measuring unit can especially be configured to be operated in the measuring mode when the sensor module comes into contact properly with a sample or a patient. For this purpose, the measuring unit, in particular the sensor station or the sensor module, can be configured to monitor the status of the sensor module in order to determine whether the sensor module is in contact with a sample or a patient. For example, the sensor module can be configured to carry out measurements within a liquid sample. The measuring unit can accordingly be configured to determine whether the sensor module, in particular a detector element of the sensor module, is immersed in a liquid or not. As soon as it is detected that the sensor module is immersed in a liquid, the measuring unit can be operated in the measuring mode. In one further embodiment, the measuring unit can be configured to change a status parameter stored in the sensor module when the sensor module comes into contact with the sample or a patient, in particular when the detector element is immersed in the liquid. More precisely, the measuring unit can provide the status parameter with a value which indicates a used status of the sensor module.

According to one further embodiment, it can be provided that at least one sensor module is configured to carry out a measurement on the patient or on a sample before it is attached to the measuring unit and is connected thereto. In other words, the sensor module can be configured to carry out a measurement in a status in which the sensor module is decoupled from the measuring unit. In response to the coupling with the measuring unit, the previously captured measurement values can then be read out by means of the measuring unit, in particular after an authentication of the sensor module has taken place, in particular as described above in conjunction with the validation mode. The authentication can be, for example, a blockchain-based authentication.

In one further embodiment, the received sensor data, which comprise or indicate the acquired measurement values, can be used by the sensor station to carry out the diagnostic analysis. In other words, the sensor station can be configured to carry out the analysis on the basis of the received sensor data and to generate an analysis result which indicates whether a disease to be detected or symptoms to be detected have been detected or not.

In the present disclosure, the term “terminal”, which can also be referred to as a user equipment, user station, or data terminal, is understood as a device which provides an access to a communication network and is accordingly attached to a network termination of the communication network or is connected thereto. The communication network can be a public or private data or telecommunication network and in particular can be provided by a computing network or a group of computing networks. Furthermore, the terminal can be connected by means of a plug connection or a radio connection to the network termination. For example, the terminal can be a computer, in particular a PC or tablet computer, a mobile telephone, a smart phone, etc. According to one configuration, the terminal can execute an application provided for this purpose to fulfill the functionalities required for the analysis system.

The terminal can be configured to ensure a data exchange between the measuring unit and the central evaluation unit. In other words, the terminal can be used as a hub for connecting the measuring unit to the evaluation unit. In particular, the terminal can ensure that an encrypted data exchange can take place between the measuring unit and the evaluation unit.

The measuring unit, in particular the terminal, can furthermore comprise a user interface. The user interface can be used to enable an interaction between a user of the measuring unit and the analysis system. The user interface can accordingly be configured to capture input information and/or output output information. According to one configuration, the measuring unit can be configured to capture application-specific information from the user, in particular in dependence on the disease or symptoms to be detected and/or in dependence on the patient or patient groups to be examined. For example, the input information can indicate which disease or which symptoms are to be detected by means of the diagnostic analysis. The information to be captured can vary in this case in dependence on the sensor module attached to the measuring unit. For example, the user interface can be configured to display a question catalog to the user and to receive the associated answers or inputs. The captured input information, in particular application-specific input information, can then be transmitted to the evaluation unit. The input information are preferably associated in this case with the measuring unit and/or the measurements carried out by means of the measuring unit and/or an analysis result. In this way, an extensive evaluation of analysis results can be carried out by means of the evaluation unit. Alternatively or additionally, the captured input information, in particular the application-specific input information, can be used as the basis of the diagnostic analysis to be carried out by means of the analysis system. In other words, the analysis can take place in dependence on the captured input information.

Alternatively or additionally, the measuring unit, in particular the terminal and/or the sensor station, can be configured to display the analysis results generated by means of the diagnostic analysis to a user of the measuring unit.

In one further embodiment, the terminal can be configured to identify the sensor module connected to the measuring unit. For this purpose, the terminal can access the sensor data, in particular a part of the sensor data, in order to read out the product identification, which enables an identification of the sensor module used. Alternatively or additionally, the terminal can be equipped with an optical sensor, for example in the form of a camera, which detects an identifier attached to the sensor module and derives the product identification therefrom. The identifier attached to the sensor module can be a barcode or a QR code.

According to one configuration, the analysis system can be configured in such a way that the access to the sensor data sent by the sensor module, in particular the access to the captured measurement values, is denied to the terminal. This can be achieved, for example, in that the measurement values captured by the sensor module are provided to the terminal in an encrypted form, but the terminal cannot carry out decryption of the measurement values. Alternatively or additionally, in the case in which the analysis is executed by the sensor station or the sensor module, an analysis result generated thereby can be passed on to the terminal, but not the measurement values captured by the sensor module.

As described above, the central evaluation unit has a communication connection to the terminal of the at least one decentralized measuring unit, in particular via the communication network, which can be a public or private data or telecommunication network and can be provided in particular by a computing network or a group of computing networks. The evaluation unit can be comprised in a web server and/or provided in the form of a cloud computing application for this purpose. The evaluation unit can accordingly have access to a web server and a database associated therewith or connected thereto.

The evaluation unit can be configured to collect and evaluate data from different decentralized measuring units in order to thus provide a centrally available data management system. In this way, large amounts of data may be managed and evaluated efficiently and effectively, by which improved control of diseases, in particular of rapidly spreading diseases, for example infectious diseases, is enabled. One advantage in this case can be that the data recorded by different measuring units are collected automatically at a central location and are thus available immediately, i.e., without significant time loss, for extensive evaluation. Accordingly, the analysis system proposed here permits a comprehensive information foundation to be provided, which is required in particular for the decision about effective measures for containing diseases.

As described above, the analysis system is configured to carry out an analysis on the basis of the measurement values measured by means of a sensor module. The function of carrying out the diagnostic analysis can be assigned to a component of the analysis system, for example the evaluation unit and/or the measuring unit. In other words, a component of the analysis system can be entrusted with the task of carrying out the analysis in order to generate the analysis result. This component entrusted with carrying out the diagnostic analysis can accordingly receive the measurement values of at least one sensor module and transmit an analysis result to the evaluation unit.

According to one embodiment, the evaluation unit can be configured to carry out the analysis. For this purpose, the evaluation unit can receive the measurement values, in particular the sensor data, and carry out the analysis on the basis thereof. In this configuration, the terminal can be used to pass on the sensor data to the evaluation unit. Furthermore, the evaluation unit can be configured to transmit an analysis result arising from the diagnostic analysis to the terminal.

Alternatively or additionally, the measuring unit, in particular the sensor station, the terminal, or the sensor module, can be configured to carry out the analysis. For this purpose, the measuring unit, in particular the sensor station, can receive the measurement values, in particular the sensor data and carry out the analysis on the basis thereof.

The diagnostic analysis can be based on a mathematical model. In other words, the analysis system, in particular the evaluation unit or the measuring unit, can be configured to carry out the analysis on the basis of a mathematical model and generate the analysis result accordingly. The mathematical model can comprise a function or can be described as such, which returns a value as a function of the captured measurement values, which indicates whether a specific disease and/or symptoms have been recognized or not. For example, a comparison of the measurement values to specified value ranges can take place by means of the mathematical model in order to determine whether the captured measurement values are in a ranges which indicate the presence of a disease or of symptoms or not.

The mathematical model underlying the diagnostic analysis can be selected or adapted for this purpose in dependence on the sensor module used and/or in dependence on the disease or symptoms to be detected. Furthermore, the evaluation unit can be configured to adapt the mathematical model underlying the diagnostic analysis, in particular on the basis of sensor data or measurement values captured up to this point and the analysis results connected thereto. In this way, central management of the mathematical model underlying the diagnostic analysis can take place, in order to ensure that the analysis based on the decentralized measurements takes place uniformly in the overall analysis system and accordingly comparability is provided between analysis results.

In one further embodiment, the evaluation unit can comprise a programming interface, which can be configured to exchange data and to interact with applications or components outside the analysis system. The evaluation unit can be configured to transmit data received via the programming interface to individual measuring units, in particular their terminals. This can take place in dependence on the measurement values captured by the measuring unit and/or the analysis results connected thereto. According to one configuration, in the case that the presence of a disease or symptoms has been established from captured measurement values, the associated measuring unit, in particular the terminal, can be supplied with information which were provided to the evaluation unit via the programming interface from a point outside the analysis system. These information can be intended for the purpose of providing a user of the measuring unit, in particular the terminal, with more extensive information on the analysis result connected thereto, in particular to assist the user in dealing with the diagnosis or during a therapeutic treatment. In the case that it has been established by means of the diagnostic analysis that a disease or accompanying symptoms have been detected in an examined patient, the data transmitted via the programming interface to the evaluation unit can comprise information on the treatment of the detected disease or symptoms, for example recommended medications or a contact point, for example a medical practice or a pharmacy, in order to enable a rapid treatment.

A sensor module is preferably embodied and configured so that the incidence and/or the concentration and/or the amount of at least two different analytes in the sample can be determined. A targeted analysis of the sample can thus be carried out by means of a single sensor module, which is reasonable for a specific diagnostic situation. For example, if a specific diagnosis is suspected, the parameters or markers relevant for this purpose can be determined in the sample simultaneously and using a single sensor module. A particularly efficient analysis can thus be carried out, because separate sensor modules do not have to be arranged in succession in the measuring unit for each of the relevant parameters or markers and the sample does not have to be measured multiple times in succession for different parameters or markers.

To be able to determine the at least two different analytes in the sample, the sensor module preferably comprises at least one separate sensor element for each analyte to be determined.

The terminal is preferably configured to provide an identifier associated with the analysis, preferably a QR code displayed on a display screen of the terminal, for a further terminal, and the evaluation unit and/or the terminal are embodied and configured so that a further terminal can authenticate itself via the identifier and can retrieve data associated with the analysis from the evaluation unit or the terminal.

In other words, a user can authenticate himself, for example, via the identifier at a later point in time and then download the analysis results to his own terminal and have them displayed there. This is useful, for example, if the analysis results first have to be released by a third person, for example a physician, before their transmission to a user.

Furthermore, a further analysis system for analyzing a sample is proposed. The further analysis system comprises a central evaluation unit and at least one decentralized measuring unit, which has a communication connection via a terminal to the evaluation unit, wherein the measuring unit is configured to accommodate different sensor modules exchangeable with one another and wherein the evaluation unit and/or the measuring unit are embodied and configured to determine the incidence and/or the concentration and/or the amount of an analyte in the sample on the basis of a measurement value measured by means of a sensor module in the sample. The evaluation unit is provided with a programming interface for receiving data associated with analysis results from a point outside the analysis system. The evaluation unit is configured to selectively transmit data received via the programming interface to individual measuring units in dependence on the measurement values captured by means of this measuring unit and/or the analysis results connected thereto.

Furthermore, an analysis system for analyzing a sample is proposed, preferably for determining chemical or biochemical information in the sample, comprising a central evaluation unit and at least one decentralized measuring unit, which has a communication connection via a terminal with the evaluation unit, wherein the measuring unit is configured to accommodate different sensor modules exchangeable with one another and wherein the evaluation unit and/or the measuring unit are embodied and configured to determine the incidence and/or the concentration and/or the amount of an analyte in the sample on the basis of a measurement value measured by means of a sensor module in the sample.

### BRIEF DESCRIPTION OF THE FIGURES

Preferred further embodiments of the invention are explained in more detail by the following description of the figures, in which:

FIG. 1 shows an analysis system according to a first embodiment; and

FIG. 2 shows an analysis system according to a second embodiment.

### DETAILED DESCRIPTION OF EMBODIMENTS

Preferred embodiments are described hereinafter on the basis of the figures. Identical, similar, or identically-acting elements are provided here with identical reference signs in the different figures, and a repeated description of these elements is partially omitted to avoid redundancies.

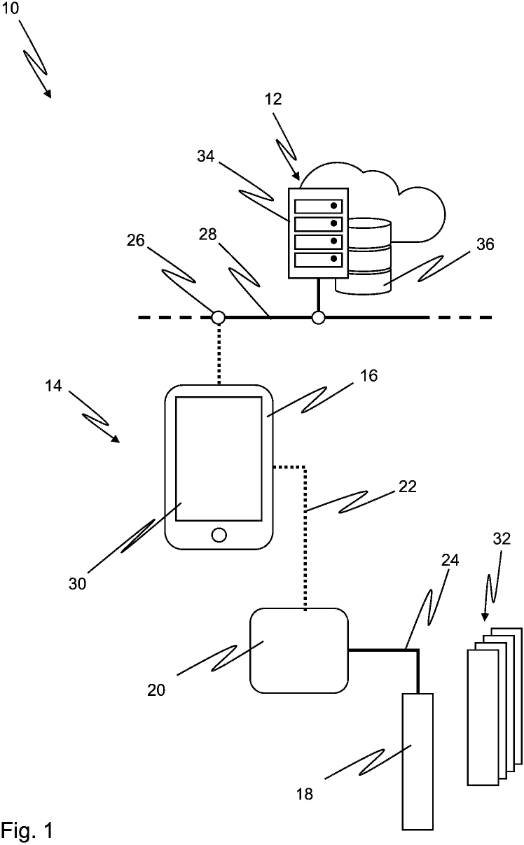

FIG. 1 shows an analysis system 10, also designated hereinafter as an “analysis system”, in particular a medical analysis system, which is configured to carry out, manage, and evaluate a large number of diagnostic analyses. More precisely, the proposed analysis system 10 permits examinations to be carried out on patients or samples in a decentralized manner, in particular at various locations, the evaluation of which is centrally controlled and/or managed, however.

The analysis system 10 can be used to detect different diseases and/or symptoms. For this purpose, the analysis system 10 forms a product platform, which is configurable to modify the analysis system 10 application-specifically. In this way, the analysis system 10 can be used to detect different diseases and/or symptoms.

To describe the structural and functional design of the analysis system 10, an possible configuration of the analysis system 10 is specified hereinafter, in which the analysis system 10 is configured to detect an infectious disease, especially to detect whether a patient is infected with a coronavirus, in particular with the SARS-CoV2 virus.

The analysis system 10 comprises a central evaluation unit 12 and at least one, preferably more than one decentralized measuring unit 14, which have a communication connection to one another via a terminal 16 of the measuring unit 14 in order to exchange data. The measuring unit 14 is configured to accommodate different sensor modules 18 which are exchangeable with one another. The evaluation unit 12 and/or the measuring unit 14 are embodied and configured to determine the incidence and/or the concentration and/or the amount of an analyte in the sample on the basis of a measurement value measured in the sample by means of one of the sensor modules 18.

The analysis system 10 is preferably configured to carry out an analysis to determine chemical or biochemical information on the basis of measurement values measured by means of a sensor module 18.

In the present embodiment, the measuring unit 14 comprises a sensor station 20, which has a communication connection to the terminal 16 via a radio connection 22 or a wired connection, in order to enable a data exchange between the terminal 16 and the sensor station 20. The radio connection 22 can be provided in particular in the form of a Bluetooth connection, a mobile radio connection, for example an LTE connection, a WLAN (Wireless Local Area Network) connection or a LoRa (Long Range Wide Area Network) connection.

The sensor station 20 is furthermore configured to be coupled with at least one sensor module 18, in particular by means of a structural and/or communication interface, in order to enable a data exchange between the sensor station 20 and the sensor module 18 connected thereto. As shown in FIG. 1, the sensor station 20 is connected by means of a wired connection 24 to at least one sensor module 18. Alternatively or additionally, at least one sensor module can be in communication exchange with the sensor station 20 via a radio connection.

As described above, the terminal 16 is provided to ensure a data exchange between the measuring unit 14 and the evaluation unit 12. For this purpose, the terminal 16 is attached via an interface 26, in particular via a network termination, to a communication network 28, to which the evaluation unit 12 is also attached. The measuring unit 14 and the evaluation unit 12 can accordingly exchange data with one another via the communication network 28. The communication network 28 can be a public or private data or telecommunication network and can be provided in particular by a computing network or a group of computing networks.

In the embodiment shown, the terminal 16 is provided in the form of a smart phone or tablet computer, but is not limited to this embodiment. Rather, the terminal 16 can be provided by any device suitable for this purpose, for example by a personal computer. The terminal 16 comprises a user interface 30, in the present case in the form of a touchscreen, which is used for interaction between a user of the terminal 16 and the analysis system 10. The terminal 16 can be provided to be operated by medically trained users, for example by medical professionals, but also by users who are not specially trained.

The analysis system 10 shown here is provided to determine different chemical or biochemical information or parameters and in particular to detect different diseases or symptoms on this basis. For this purpose, the analysis system 10 is embodied as configurable. More precisely, in the embodiment shown here the measuring unit 14 is embodied as configurable in that it is configured to accommodate different sensor modules 18 which are exchangeable with one another and connect them accordingly to the sensor station 20.

The selection of the sensor modules 18 to be used in the measuring unit 14 can take place in dependence on the information, features, or parameters to be determined and/or in dependence on the disease to be detected and/or the symptoms to be detected. For this purpose, the analysis system 10 comprises a set 32 of different sensor modules 18 which are exchangeable with one another. The sensor modules 18 contained in the set 32 differ in this case with regard to their suitability for determining specific chemical or biochemical information with respect to an analyte in a sample and/or for detecting specific diseases and/or symptoms.

In particular, the different sensor modules 18 can each comprise specific sensor elements for determining different specific chemical or biochemical information.

In other words, each sensor element can output a separate measurement value, which is then transmitted to the decentralized measuring unit and/or the terminal and/or the central evaluation unit. Accordingly, if the sensor module 18 has two sensor elements, also two different measurement values are measured and transmitted. Alternatively or additionally, the evaluation unit 12 and/or the terminal 16 can be configurable with respect to an item of chemical or biochemical information to be determined and/or can be configurable specifically by disease and/or symptom, both structurally, in that individual hardware components can be exchanged or adapted, or functionally, for example in that software components can be exchanged or adapted.

For example, specific sensor elements of the sensor modules 18 can be selectable with regard to chemical or biochemical information to be determined or specifically by disease and/or symptom, in order to configure a suitability of the respective sensor module 18 for the respective application.

For example, a first sensor module 18 can be configured with regard to chemical or biochemical information to be detected, in particular configured specifically by disease and/or symptom, by the selection of a first subgroup of sensor elements accommodated in the first sensor module 18.

A second sensor module 18, which is physically identical per se to the first sensor module 18, can then be configured for different chemical or biochemical information to be detected by the selection of a second subgroup of sensor elements accommodated in the sensor module, which is different from the first subgroup, in particular can be configured for a different disease-specific and/or system-specific question.

The selection of the sensor elements of the sensor module 18 can be performed by circuitry, for example via different contacting or the switching off and on of specific contacts, and/or in software for the respective configurations.

The specific selection of the sensor elements accommodated in the sensor module 18 can accordingly be achieved, for example, in that only the sensor elements of the subgroup are contacted and no contact is established to the other sensor elements. This can be achieved, for example, via a contacting specific for the respective application - for example via a specific plug - which only contacts the sensor elements of the subgroup. The specific selection and the accompanying contacting can also be achieved via corresponding switches, which are only closed for the sensor elements of the subgroup and remain open for the other sensor elements. The switch configuration required for the respective application can be actuated via software or a fixed specification.

The selection is preferably implemented by the decentralized measuring unit, which has the corresponding switch and plug devices. The actuation of the switch and plug devices can take place from the decentralized measuring unit and/or from the central evaluation unit and/or from the terminal.

In this embodiment, in which contacting of one subgroup of the sensor elements takes place, the measurement values are only captured for the actually contacted sensor elements. Accordingly, no measurement values can be captured by the remaining sensor elements.

The selection of the subgroup of the sensor elements can also take place on the evaluation side, wherein measurement values are captured by all sensor elements and then only those of the subgroup for the respective specific evaluation are used by means of filtering of the measurement values.

For example, an analysis system 10 is proposed for analyzing a sample, preferably for determining chemical or biochemical information in the sample, comprising a central evaluation unit 12 and at least one decentralized measuring unit 14, which has a communication connection via a terminal 16 to the evaluation unit 12, wherein the measuring unit 14 is configured to accommodate different sensor modules 18 exchangeable with one another and wherein the evaluation unit 12 and/or the measuring unit 14 are embodied and configured to determine the incidence and/or the concentration and/or the amount of an analyte in the sample on the basis of a measurement value measured in the sample by means of a sensor module 18.

At least one sensor module 18 can have at least two sensor elements here for determining one measurement value each and the sensor module 18 can be configured to transmit the at least two measurement values of the sensor module 18 to the decentralized measuring unit 14 and/or the terminal 16 and/or the central evaluation unit 12.

The sensor module 18 and/or the decentralized measuring unit 14 and/or the terminal 16 and/or the central evaluation unit 12 can be configured to select one subgroup of the sensor elements of the sensor module 18 in order to carry out an application-specific analysis.

In the configuration shown here, the sensor module 18 used in the measuring unit 14 is configured for virus detection in a sample. A sample designates in this case a limited amount of a substance which was taken from a larger amount of the substance, for example from a reservoir, wherein the composition of the sample is representative of the composition of the substance in the reservoir and accordingly the corresponding incidence in the reservoir can be concluded from the substance incidence and the substance composition of the sample. For example, a sample can be a saliva sample or a blood sample or a swab, in particular a throat swab or a nasal swab or a sinus swab, or removed tissue.

The sensor module 18 is especially provided for detecting viruses, in particular coronaviruses, for example the SARS-CoV2 virus. In other words, the sensor module 18 is configured to carry out an examination on the sample and to acquire measurement values in this case, on which the analysis is based. In the diagnostic analysis, it is then determined on the basis of the measurement values captured by the sensor module 18 whether a virus to be detected is contained in the sample or not.

The virus in this case is the substance, the presence of which in the sample is to be qualitatively and/or quantitatively established or detected with the aid of the sensor module. The virus can in particular be directly present in the sample or can adhere to a part of the sample, in particular a sample particle. Generally formulated, the sample contains chemical or biochemical information about the virus. The chemical and/or biochemical information can comprise, for example, the incidence or the concentration of the virus.

The sensor module 18 is configured to convert the chemical and/or biochemical information contained in the sample, relating to the virus to be detected, into an electrical signal, wherein the electrical signal corresponds to the measurement values captured by means of the sensor module 18. For example, the measurement values can indicate a voltage measured by means of the sensor module 18 during the examination of the sample over time. The analysis to be carried out by means of the analysis system is accordingly configured to determine on the basis of these measurement values whether the virus is present in the sample, in order to thus detect an illness of a patient associated with the sample.

A specific embodiment of the sensor module 18, which is configured for detecting viruses, in particular coronaviruses, for example the SARS-CoV2 virus, is described in more detail below.

The procedure and the interaction of individual components of the analysis system 10 when carrying out the diagnostic analysis are described hereinafter, by means of which the analysis system 10 is provided with the capability of detecting a virus infection of a patient. In other words, the analysis system 10 is configured to carry out analyses in order to assess whether a virus or its chemical traces are present in the sample and accordingly whether a patient is affected by a virus infection or not.

The analysis takes place on the basis of the measurement values captured by the sensor module 18. In the embodiment shown here, the analysis as such, more precisely the interpretation of the measurement values captured by the sensor module 18 to detect the disease, is carried out by the terminal 16, which is equipped for this purpose with a corresponding software program and a corresponding analysis algorithm.

The sensor module 18 is configured for this purpose to transmit the data indicating measurement values to the sensor station 20, which thereupon processes the data thus received, in particular encrypts them, or transmits them unprocessed to the terminal 16. This takes place by means of the wired connections 24 and the radio connection 22 or a wired connection, via which the information designated as sensor data are transmitted from the sensor module 18 to the sensor station 20 and thereupon to the terminal 16. Different information can be transmitted by means of the sensor data. More precisely, the sensor data are provided for the purpose of transmitting the measurement values captured by means of the sensor module 18, data specifying the sensor module 18, and measurement data, wherein the measurement data specify the measurement executed by the sensor module 18 to capture the measurement values.

Carrying out the measurements and transmitting the sensor data connected thereto by means of the sensor module 18 presume that the sensor module 18 is properly coupled to the measuring unit 14, in particular the sensor station 20.

The coupling of the sensor module 18 to the measuring unit 14, in particular the sensor station 20, is described hereinafter. In a first step, a user of the measuring unit 14 connects the sensor module 18 provided for the measurement to the sensor station 20 via the wired connections 24 by means of a structural interface provided for this purpose.

The sensor station 20 is configured to detect whether a structural or physical coupling of the sensor module 18 to the sensor station 20 takes place or exists. In response thereto, the sensor station 20 is operated in an initialization mode, in which the sensor station 20 identifies the sensor module 18 connected thereon. This takes place on the basis of information which the sensor module 18 transmits by means of the sensor data to the sensor station 20. For this purpose, the sensor module 18 can send a product identification number to the sensor station 20, by means of which an identification and authentication can take place by way of the sensor station 20. Alternatively, an identification and authentication of the sensor module 18 can take place with the aid of the terminal 16 in that, for example, a user transmits an item of information identifying the sensor module 18 via the user interface 30, which is transmitted to the sensor station 20 via the radio connection 22. A sensor of the terminal 16, for example a camera, can also be used for the purpose of reading out identification information from the sensor module 18, for example from a QR code applied thereto, which can also be transmitted via the radio connection 22 to the sensor station 20.

As soon as an identification and authentication have taken place, the proper status of the sensor module is checked. For this purpose, the sensor station 20 is operated in a validation mode, in which the sensor station 20 causes the sensor module 18 to carry out a reference measurement. During the reference measurement, the sensor module 18 is preferably not in contact with a sample or liquid. In other words, during the reference measurement the sensor module 18 can be in contact with a reference medium specified for the reference measurement, for example air or an inert gas. During the reference measurement, the sensor module 18 captures reference measurement values, which are transmitted to the sensor station 20 via the wired connection 24, in particular in the form of a magnetic plug connection. After receiving the reference measurement values, the sensor station 20 compares them to a specified value range in order to determine whether the sensor module 18 has a proper status or not. Alternatively or additionally, the sensor station 20 can be configured in the validation mode to query a status of the sensor module 18, in particular whether it is in an unused or in a used state. For this purpose, the sensor module 18 can transmit a status parameter to the sensor station 20. The examination of a sample by means of the sensor module 18 can only take place in a next step when the status parameter corresponds to a predetermined status, for example the unused status, and/or the proper status of the sensor module 18 has been determined. Alternatively, the identification and authentication and/or the check of the proper status of the sensor module 18 can take place by means of the terminal 16.

To carry out the examination of the sample, i.e., to carry out the measurements by means of the sensor module 18, the sensor station 20 is operated in a measuring mode. In the embodiment shown here, the sensor station 20 is configured to change into the measuring mode as soon the sensor module 18 comes into contact with the sample to be examined, more precisely when a sensor element of the sensor module 18 is immersed in a sample liquid. For this purpose, the sensor station 20 monitors the signal output of the sensor module 18, i.e., the measured measurement signals, which change according to a defined manner known to the sensor station as soon as the sensor module 18 is immersed in a sample liquid. In the measuring mode, the sensor station 20 is configured to record the measurement values captured by the sensor module 18 during the examination of the sample and/or to receive them for further processing from the sensor module 18.

The terminal 16 is configured, as described above, to carry out the analysis on the basis of the measurement values in order to determine whether a patient to be associated with the sample is affected by the viral disease or not. For this purpose, the terminal 16 makes use of a mathematical model, in particular in the form of an algorithm executed in the sensor station. The management of the mathematical model is incumbent upon the evaluation unit 12, which can cause an adaptation or modification of the mathematical model. The mathematical model can be selected application-specifically and provided to the sensor station 20 by means of the evaluation unit 12. This can take place in dependence on the sensor module 18 attached to the sensor station 20.

As a result of the diagnostic analysis, the terminal 16 generates an analysis result, which indicates whether the patient associated with the examined sample is affected by the viral disease or not. The analysis result is then transmitted with the aid of the terminal 16, i.e., via the radio connection 22 and the communication network 28, to the evaluation unit 12, which manages and evaluates the analysis results of all examinations performed with the aid of the different measuring units 14 in the analysis system 10. In one further embodiment, the analysis result can be displayed to the user of the measuring unit 14 via the terminal 16 or the sensor station 20 or a further terminal (not shown here).

The further terminal can be a terminal associated with the patient, for example, a tablet computer, a mobile telephone, a smart phone, etc. The further terminal can comprise a software program, which is provided for the communication with the analysis system 10, in particular with the terminal 16 and/or the evaluation unit 12. In order to enable a retrieval of the analysis result or establish the communication with the analysis system 10, the terminal 16 can be configured to provide an identifier associated with the analysis for the further terminal. This identifier can be, for example, a QR code, which can be displayed on the display screen of the terminal 16 or transmitted to the further terminal by the terminal. The further terminal can thus use the transmitted identifier for the purpose of authenticating itself in relation to the evaluation unit 12 and/or the patient and retrieving data associated with the analysis, for example the analysis result, from the evaluation unit 12 or the terminal 16.

The data exchange via the wired connection 24, the radio connection 22, which can also be embodied as a wired connection, and the communication network 28 preferably takes place in encrypted form. Accordingly, at least the evaluation unit 12, the sensor module 18, and the sensor station 20 can be equipped with means for encrypting and decrypting data.

The evaluation unit 12 is comprised in a web server 34 and provided in the form of a cloud computing application. The evaluation unit 12 accordingly has access to the web server 34 and a database 36 associated therewith or connected thereto.

The evaluation unit 12 is configured to collect and evaluate data from different decentralized measuring units 14 in order to thus provide a centrally available data management system. The evaluation unit 12 comprises a programming interface, which is configured to exchange data and to interact with applications or components outside the analysis system 10. The evaluation unit 12 is configured to transmit received data to individual measuring units 14, in particular their terminals 16, via the programming interface. This takes place in dependence on the measurement values captured by the measuring unit 14 and/or the analysis results connected thereto. According to one configuration, in the case in which the presence of a disease or of symptoms has been established for captured measurement values, the associated measuring unit 14, in particular the terminal 16, can be supplied with information which were provided to the evaluation unit 12 via the programming interface from a point outside the analysis system. These information can be provided for the purpose of providing the user of the measuring unit 14, in particular the terminal 16, with more extensive information on the analysis result connected thereto, in particular to assist the user in dealing with the diagnosis or in a therapeutic treatment. In the case that it has been established by means of the analysis that a disease or accompanying biochemical or chemical information, which indicate disease, have been detected in an examined patient, the data transmitted via the programming interface to the evaluation unit 12 can comprise information on the treatment of the detected disease or symptoms such as recommended medications or a contact point, for example a medical practice or a pharmacy, to enable a rapid treatment.

An embodiment of the sensor module 18 is described hereinafter, which can be used for detecting viruses, in particular coronaviruses, for example the SARS-CoV2 virus, in the above-described analysis system 10.

In general, the sensor module 18 is configured to convert the chemical and/or biochemical information contained in the sample into an electrical signal. For this purpose, the sensor module 18 comprises a sensor element, which is configured to convert chemical and/or biochemical information, relating to a virus in the sample, into an electrical signal. The sensor element especially comprises a cantilever, also designated as a spring element, which has a rigid base and a deflectable part, wherein a receptor layer for selectively accommodating a virus to be detected from the sample is applied on the deflectable part. A passive test transducer is arranged on the rigid base and an active test transducer is arranged on the deflectable part.

The cantilever is configured to interact with the virus in the sample in order to thus derive the biochemical information in the sample via a change of the cantilever. The cantilever is in this case a spring element, which has a rigid base and a deflectable part. The rigid base is accordingly an immovable part of the cantilever which is in particular arranged fixed in place on a substrate. The deflectable part of the cantilever is arranged on the rigid base and protrudes beyond the substrate on which the rigid base is arranged. If the cantilever is bent, material stresses and forces thus result in or on the material of the cantilever, which can be measured. If such a material stresses, in particular surface stresses can be measured, a deflection or bending of the cantilever may be derived from this.

The transducers have the purpose of determining or measuring the bending of the cantilever induced by the change of the surface stress. The active transducer is arranged on the deflectable part of the cantilever, whereas the passive transducer is arranged on the rigid base of the cantilever. In particular, electrical properties of a circuit can be influenced via the transducers. For example, bending of the cantilever can have the result that the resistance of a transducer, for example of the active transducer, increases, while no bending of the cantilever also induces no change of the resistance of the transducer. This can take place, for example, via an embodiment of the transducers according to the principle of a strain gauge, due to which bending of the respective cantilever is expressed in a length change of the strain gauge of the transducer applied thereon and thus bending of the cantilever can be detected directly by a change of the resistance of the strain gauge. The chemical and/or biochemical information in the sample is thus detectable via bending of the cantilever, subsequent registration via a transducer, and via a change of an electrical property of a circuit. In that the active transducer is arranged on the deflectable part of the cantilever, a measure can thus be found which corresponds to the strength of interaction of the virus with the deflectable part. In contrast, the passive transducer is arranged on the rigid base, so that the interaction is reduced to those interactions which do not primarily induce a deflection of the deflectable part.

Due to the binding of the virus on the receptor layer, a force acts on the cantilever. The force on the cantilever is larger, for example, the larger the concentration of the virus is in the sample or the larger the incidence is in the sample. A receptor layer is in this case a substance which can interact with the virus. Interaction in this case means that the virus is in chemical and/or biochemical and/or physical interaction with the receptor layer. This in turn means that the receptor layer is selected specifically for the virus to be detected.

To achieve this effect, the receptor layer comprises antibodies for an antigen, i.e., for protein fragments (proteins) of the virus to be detected. Antibodies are proteins which are produced by T cells as a reaction product to antigens. Antibodies are typically used by the human immune system for the purpose of binding to the antigens of viruses, so that the viruses can be marked and an outbreak of a virus infection can be prevented by the immune system. In this way, it is made possible for the receptor layer, more precisely the antibodies comprised therein, to interact with the virus, in particular to bind thereto, which results in bending of the cantilever, in particular due to change of the surface stress on the cantilever.

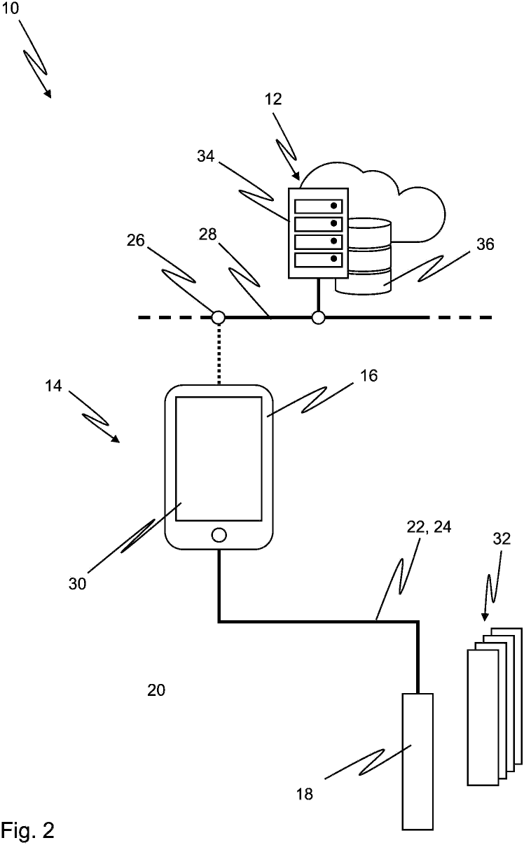

FIG. 2 shows a further embodiment of the analysis system 10, in which the evaluation unit 12 is configured to execute the analysis on the basis of the measurement values captured by the sensor module 18. The measuring unit 14 is accordingly configured to transmit the measurement values captured by a sensor module 18 to the evaluation unit 12. This takes place via the terminal 16, via which the sensor module 18 and the evaluation unit 12 are in communication exchange.

Sensor modules 18 which are exchangeable with one another can be coupled with the terminal 16, in particular directly with the terminal 16, in the measuring unit 14 according to this configuration of the analysis system 10, as shown in FIG. 2. For this purpose, at least one sensor module 18 can be connected to the terminal 16 via a radio connection 24 or a wired connection 22. In contrast to the configuration shown in FIG. 1, the measuring unit 14 shown here according to the further embodiment can be equipped without a sensor station 20. The functions of the sensor station 20, as described in conjunction with the configuration shown in FIG. 1, can accordingly be allocated to the evaluation unit 12 and optionally partially to the terminal 16 or another component of the measuring unit 14, for example to the sensor module 18. Alternatively, the functions of the sensor station 20 can be allocated completely to the terminal 16 or another component of the measuring unit 14, for example to the sensor module 18. In the configuration shown here, the function of the execution of the diagnostic analysis, as already described above, is allocated to the evaluation unit 12. The further functions, such as the coupling of the sensor module 18 with the measuring unit 14 and the validation of a proper status of the sensor module 18, can be allocated to the terminal 16 or the evaluation unit 12 or can be executed by the sensor module 18 itself.

According to one further embodiment, the analysis system 10 can be configured to be connected both to measuring units according to the embodiment shown in FIG. 1 and also to a measuring unit according to the embodiment shown in FIG. 2. It can be decided in this case application-specifically and/or in dependence on the structural embodiment of the measuring unit 14 whether the analysis is executed by means of the evaluation unit 12, the sensor station 20, or another component of the analysis system 10.

If applicable, all individual features which are represented in the exemplary embodiments can be combined with one another and/or exchanged, without leaving the scope of the invention.

List of reference numerals

| | |
|---|---|
| 10 | analysis system |
| 12 | evaluation unit |
| 14 | measuring unit |
| 16 | terminal |
| 18 | sensor module |
| 20 | sensor station |
| 22 | radio connection |
| 24 | wired connection |
| 26 | interface to communication network |
| 28 | communication network |
| 30 | user interface |
| 32 | set of different sensor modules exchangeable with one another |
| 34 | web server |
| 36 | database |

What is claimed:

1. An analysis system for analyzing a sample, the system comprising:
a central evaluation unit;
a plurality of sensor modules that are exchangeable with one another; and
at least one decentralized measuring unit having a communication connection via a terminal to the evaluation unit and being configured to accommodate the plurality of sensor modules that are exchangeable with one another,
wherein at least one of the evaluation unit and the at least one decentralized measuring unit are configured to determine at least one of an incidence, a concentration and an amount of an analyte in the sample based on a measurement value measured in the sample by at least one of the sensor modules, and
wherein the plurality of sensor modules differ with regard to a respective suitability for determining at least one of chemical and biochemical information of the analyte in the sample.

2. The analysis system according to claim 1, wherein the plurality of sensor modules are each configured to determine different chemical or biochemical information, which are respective chemical or biochemical indicators for diseases or symptoms.

3. The analysis system according to claim 2, wherein the respective chemical or biochemical indicators are for detecting disease-specific features or parameters for detecting a viral infection.

4. The analysis system according to claim 1, wherein the at least one of the plurality of sensor modules is configurable with regard to the chemical or biochemical information to be detected by a selection of a subgroup of sensor elements accommodated in the at least one sensor module.

5. The analysis system according to claim 1, wherein the at least one of the plurality of sensor modules is configured to transmit sensor data to the measuring unit that includes at least one of the measurement value measured by the sensor module, data specifying the sensor module, and data specifying a measurement carried out by sensor module.

6. The analysis system according to claim 1, wherein the measuring unit further comprises a sensor station that has a communication connection to the terminal and is configured to be coupled with the at least one sensor module.

7. The analysis system according to claim 1, wherein the measuring unit is configured to:
operate in an initialization mode in which the measuring unit is configured to identify an accommodated sensor module and to check by a QR code whether the sensor module is authenticated for operation with the measuring unit (14), or
operate in a validation mode in which the measuring unit is configured to detect a proper status or operation of the sensor module, or
operate in a measuring mode in which the sensor module is controlled to capture the measurement value, with the measuring unit changing into the measuring mode when the sensor module comes into contact with a sample or a patient.

8. The analysis system according to claim 1, wherein the terminal is connected via a communication network to the evaluation unit, and the evaluation unit is comprised in a web server.

9. The analysis system according to claim 1, wherein the terminal comprises a user interface that is configured to capture application-specific input information from a user of the measuring unit, and the terminal is configured to transmit the application-specific input information to the evaluation unit.

10. The analysis system according to claim 1,
wherein at least one of the evaluation unit and the measuring unit is configured to execute an analysis based on a mathematical model, and
wherein the central evaluation unit is configured to adapt the mathematical model based on previously captured measurement values and analysis results connected thereto.

11. The analysis system according to claim 1, further comprising:
the central evaluation unit and at least one decentralized measuring unit that has a communication connection via a terminal to the evaluation unit,
wherein the measuring unit is configured to accommodate the plurality of sensor modules that are exchangeable with one another,
wherein at least one of the evaluation unit and the measuring unit is configured to determine the at least one of the incidence, the concentration, and the amount of an analyte in the sample based on a measurement value measured in the sample by the sensor module, and
wherein the evaluation unit includes a programming interface for receiving data associated with analysis results from a point outside the analysis system and is configured to transmit data received via the programming interface selectively to individual measuring units in dependence on the measurement values captured by the measuring unit or the analysis results connected thereto.

12. The analysis system according to claim 11, wherein the at least one of the plurality of sensor modules is configured to determine the at least one of the incidence, the concentration, and the amount of at least two different analytes in the sample.

13. The analysis system according to claim 12, wherein the terminal is configured to provide an identifier associated with the analysis results that is displayed on a display screen of the terminal, and at least one of the evaluation unit and the terminal is configured so that an additional terminal is configured to authenticate itself via the identifier and retrieve the data associated with the analysis results from the evaluation unit or the terminal.

14. An analysis system for determining chemical or biochemical information in a sample, the system comprising:
a central evaluation unit; and
at least one decentralized measuring unit that has a communication connection via a terminal to the central evaluation unit and is configured to accommodate a plurality of sensor modules that are exchangeable with one another,
wherein at least one of the evaluation unit and the measuring unit is configured to determine at least one of an incidence, a concentration, and an amount of an analyte in the sample based on a measurement value measured in the sample by at least one of the sensor modules.

15. The analysis system according to claim 14, wherein the at least one of the sensor modules has at least two sensor elements that are each configured to determine one measurement value and the at least one sensor module is configured to transfer the at least two measurement values to at least one of the decentralized measuring unit, the terminal, and the central evaluation unit.

16. The analysis system according to claim 15, wherein the at least one of the sensor modules is configured to select a subgroup of the at least two sensor elements in order to execute an application-specific analysis.

17. The analysis system according to claim 15, wherein the decentralized measuring unit is configured to select a subgroup of the at least two sensor elements in order to execute an application-specific analysis.

18. The analysis system according to claim 15, wherein the terminal unit is configured to select a subgroup of the at least two sensor elements in order to execute an application-specific analysis.

19. The analysis system according to claim 15, wherein the central evaluation unit is configured to select a subgroup of the at least two sensor elements in order to execute an application-specific analysis.
